(19) United States
(12) Patent Application Publication
(10) Pub. No.: US 2020/0069607 A1
(43) Pub. Date: Mar. 5, 2020

(54) MOUTHWASH FOR TREATING ORAL CANCERS

(71) Applicant: THE BOARD OF REGENTS OF THE UNIVERSITY OF TEXAS SYSTEM, Austin, TX (US)

(72) Inventor: Cara B. GONZALES, San Antonio, TX (US)

(73) Assignee: THE BOARD OF REGENTS OF THE UNIVERSITY OF TEXAS SYSTEM, Austin, TX (US)

(21) Appl. No.: 16/466,447

(22) PCT Filed: Nov. 22, 2017
(86) PCT No.: PCT/US2017/063129

§ 371 Date: Jun. 4, 2019

Related U.S. Application Data
(60) Provisional application No. 62/431,181, filed on Dec. 7, 2016.

(51) Int. Cl. A61K 31/05; A61K 36/53; A61K 9/46; A61P 35/00

(52) U.S. Cl. CPC A61K 31/05; A61P 35/00; A61K 9/0007; A61K 36/53

(57) ABSTRACT

Provided are oral delivery compositions, including mouthwashes, for the delivery of thymol to a subject at risk of or having oral cancer.

This application claims benefit of priority to U.S. Provisional Application Ser. No. 62/431,181, filed Dec. 7, 2017, the entire contents of which are hereby incorporated by reference.

### BACKGROUND

### I. Field

The present disclosure concerns compositions and combinations for decreasing the risk of or treating oral cancers. More specifically, the disclosure relates to methods and compositions for the prevention or treatment of oral cancers such as squamous cell carcinomas using thymol- and thymol derivative-containing solutions such as mouthwashes.

### II. Related Art

Head and neck squamous cell carcinoma (HNSCC) is the sixth most common cancer in the United States with a poor overall survival rate of merely 50% (1). This is due primarily to late-stage disease that is refractive to conventional therapies including surgical resection, platinum based chemotherapy and/or radiation therapy (1). Therefore there is a great need to develop new drugs to eradicate this deadly disease.

### SUMMARY

Thus, in accordance with the present disclosure, there is provided a method of preventing or treating an oral cancer in a subject comprising administering isolated thymol or derivative thereof to an oral cavity of said subject. The oral cancer may be a squamous cell carcinoma or adenocarcinoma. The subject may be a non-human mammal, or a human, such as where the human subject partakes in one or more of smoking, chewing tobacco or consuming alcoholic beverages, such as on regular/chronic basis. The thymol or derivative thereof may be administered in a mouthwash, which may further comprise one or more of a flavorant, a preservative, fluoride or an anti-bacterial/anti-fungal agent. In particular, the mouthwash may be alcohol free. The thymol or derivative thereof may be delivered in a tablet, such as an effervescent tablet.

The thymol or derivative thereof may be administered at about 1 to 100 mM, about 1-50 mM, about 1-20 mM, about 1-10 mM, or about 2-5 mM. The thymol or derivative thereof may be administered more than once, such as daily, twice daily, or every other day. The subject may be known to have an oral HPV infection. The subject may have previously been diagnosed with oral cancer, or may not previously have been diagnosed with oral cancer. The thymol or derivative thereof may be injected into a tumor.

The method may further comprise administering to said subject a second cancer therapy, such as chemotherapy, radiotherapy, immunotherapy, toxin therapy, phototherapy, cryotherapy or surgery. The method may result in the inhibition of cancer development, cancer recurrence, cancer metastasis, cancer growth or cancer progression. The method may result in the reduction of tumor size, tumor invasion, tumor spread, tumor recurrence or patient discomfort. The method may result in an unresectable tumor being rendered resectable, or may result in an increase in a subject's survival.

Also provided is a mouthwash comprising thymol and a flavorant, wherein said flavorant is not thymol. Also provided is an effervescent tablet comprising thymol, and optionally including a flavorant, wherein said flavorant is not thymol. Also provided is an aqueously soluble powder comprising thymol, and optionally including a flavorant, wherein said flavorant is not thymol. Also provided is a kit comprising a mouthwash, effervescent tablet or powder comprising thymol and a flavorant, wherein said flavorant is not thymol, which kit may further comprise instructions for the use of said mouthwash, effervescent tablet or powder for the prevention and/or treatment of cancer.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the disclosure, and vice versa. Furthermore, compositions and kits of the disclosure can be used to achieve methods of the disclosure.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The disclosure may be better understood by reference to one or more of these drawings in combination with the detailed description of specific embodiments presented herein.

FIGS. 1A-B: (FIG. 1A) Immunofluorescent staining of TRPA1 (red), TRPM8 (red), and Vimentin (green) in cancer cell lines (20X); Top Row: Ca127 OSCC cell line. Bottom Row: HeLa cervical cancer cell line. (FIG. 1B) RT-PCR of TRPA1 mRNA in normal oral keratinocytes (NOK) and HeLa cells.

FIGS. 2A-B: Cell Proliferation assays of Ca127 (OSCC) cells (FIG. 2A) and HeLa (Cervical cancer) cells (FIG. 2B) treated with thymol for 24 and 48 hours respectively; n=4 per group, **p<0.01, ***p<0.001.

FIGS. 3A-D: Clonogenic Assay of Ca127 (FIGS. 3A and 3C) and HeLa cells (FIGS. 3B and 3D). A single two minute exposure to thymol (4.3 mM) abolished colony formation for ten days.

FIGS. 4A-B: (FIG. 4A) Tumor volumes of Ca127 xenografts treated with thymol (4.3 mM) every other day for 26 days. Significant reduction is seen by day 16 (n=6, ***p<0.001). (FIG. 4B) Scatter plot of tumor volumes at Day 26. Median tumor volume for control tumors is 400 mm³ whereas median tumor volume for thymol treated tumors is 100 mm³.

FIGS. 5A-B: (FIG. 5A) Tumor volumes of HeLa xenografts treated with thymol (4.3 mM) every other day for 22 days. Significant reduction is seen by day 18 (n=8, ***p<0.001). (FIG. 5B) Scatter plot of tumor volumes at Day 26. Median tumor volume for control tumors is 900 mm³ whereas median tumor volume for thymol treated tumors is 500 mm³.

FIGS. 6A-D: Calcium imaging of CHO cells overexpressing TRPA1 (FIG. 6A) or TRPV1 (FIG. 6B). Calcium imaging of cancer cell lines, Ca127 (FIG. 6C) and HeLa (FIG. 6D).

FIGS. 7A-B: Calcium imaging of Ca127 cells pre-treated with TRPA1 inhibitor HC030031 followed by thymol (400 μM; FIG. 7A) or with the TRPM8 inhibitor RQ00203078 followed by thymol (400 μM; FIG. 7B).

FIG. 8: Western blot analysis of cleaved PARP (c-PARP) in cancer cell lines treated with increasing concentrations of thymol.

### DETAILED DESCRIPTION

Given that greater than 60% of FDA-approved anti-cancer drugs are natural products, semisynthetic compounds derived from a natural product, or a synthetic product developed based upon the pharmacophore of natural products (2-4), the inventor screened the anti-proliferative effects of 1,088 extracts obtained from 321 plants indigenous to South Texas against numerous cancer cell lines including colon, prostate, breast and cervical cancers. An extract of the leaves and stems of Monarda citriodora, Horsemint/Lemon Beebalm, yielded a fraction with significant cytotoxicity in all cell lines tested. The active compound in this fraction was determined to be thymol, a phenolic compound found in the essential oil of many plants including thyme, oregano, rosemary, bay laurel leaves, mandarin oranges, and cranberries (5-9). Thymol is shown to have antimicrobial, anti-inflammatory and anti-oxidant properties and is found commercially in numerous products including cosmetics, pharmaceutical preparations and mouth rinses (6, 10-13). Given that thymol is already used in over-the-counter oral rinses, the inventor evaluated its anti-proliferative/anti-tumor effects against HNSCC using cell based assays and mouse xenograft models.

Additionally, thymol is a known TRP channel agonist that activates TRPA1 and weakly, TRPM8 channels allowing the influx of cations, preferentially calcium, into the cell (14-16). TRP channel expression is shown to be differentially expressed in numerous tumor types however their role in tumorigenesis remains largely unknown (14, 17-21). It is hypothesized that activation of TRP channels in cancers will result in a large influx of Ca′ and subsequent Ca′ mediated apoptosis thereby making these channels potential therapeutic targets. Indeed, studies evaluating the effect of thymol on calcium homeostasis and viability in human glioblastoma cells demonstrated an increase in Ca²⁺ levels that coincided with thymol cytotoxicity (22). However this cytotoxicity was not abolished by Ca²⁺ chelation implicating another mechanism-of-action. These studies were designed to evaluate if thymol cytotoxicity involves TRP channel activation in HNSCC or if it is due to an alternate mechanism-of-action. These and other aspects of the disclosure are set out in detail below.

### I. ORAL CANCERS

Head and neck cancer is a group of cancers that starts within the mouth (oral cancer), nose, throat, larynx, sinuses, or salivary glands. Symptoms may include a lump or sore that does not heal, a sore throat that does not go away, trouble swallowing, or a change in the voice. There may also be unusual bleeding, facial swelling, or trouble breathing.

About 80% of head and neck cancer is due to the use of alcohol or tobacco. Other risk factors include betel quid, certain types of human papillomavirus, radiation exposure, certain workplace exposures, and Epstein-Barr virus. Head and neck cancers are most commonly of the squamous cell carcinoma type. The diagnosis is confirmed by tissue biopsy. The degree of spread may be determined by medical imaging and blood tests.

Prevention is by not using tobacco or alcohol. While screening in the general population does not appear to be useful, screening high risk groups by examination the throat might be useful. Often head and neck cancer is curable if detected early; however, outcomes are typically poor if detected late. Treatment may include a combination of surgery, radiation therapy, chemotherapy, and targeted therapy. Following treatment of one head and neck cancer people are at higher risk of a second cancer.

In 2013 head and neck cancers globally affected more than 4.6 million people (mouth 2.4 million, throat 1.3 million, larynx 0.9 million) and resulted in more than 362,000 deaths (mouth 135,000, throat 139,000, larynx 88,000). Together they are the seventh most frequent cancer and the ninth most frequent cause of death from cancer. In the United States about one percent of people are affected at some point in their life and males are affected twice as often as females. The usually age at diagnosis is between 55 and 65 years. The average 5-year survival following diagnosis in the developed world is 42 to 64%.

Squamous cell cancers are common in the mouth, including the inner lip, tongue, floor of mouth, gingivae, and hard palate. Cancers of the mouth are strongly associated with tobacco use, especially use of chewing tobacco or “dip”, as well as heavy alcohol use. Cancers of this region, particularly the tongue, are more frequently treated with surgery than are other head and neck cancers.

Surgeries for oral cancers include Maxillectomy (can be done with or without orbital exenteration); Mandibulectomy (removal of the mandible or lower jaw or part of it); Glossectomy (tongue removal, can be total, hemi or partial); Radical neck dissection; Mohs procedure; and combinational, e.g., glossectomy and laryngectomy done together. The defect is typically covered/improved by using another part of the body and/or skin grafts and/or wearing a prosthesis.

When DNA undergoes oxidative damage, two of the most common damages change guanine to 8-hydroxyguanine or to 2,6-diamino-4-hydroxy-5-formamidopyrimidine. Alcohol and tobacco play a significant role. More than 75% of cases are believed to be due to these two factors.

Tobacco smoke is one of the main risk factors for head and neck cancer and one of the most carcinogenic compounds in tobacco smoke is acrylonitrile. Acrylonitrile appears to cause DNA damage indirectly by increasing oxidative stress, leading to increased levels of 8-oxo-2′-deoxyguanosine (8-oxo-dG) and formamidopyrimidine in DNA (see image). Both 8-oxo-dG and formamidopyrimidine are mutagenic. DNA glycosylase NEIL1 prevents mutagenesis by 8-oxo-dG and removes formamidopyrimidines from DNA.

However, cigarette smokers have a lifetime increased risk for head and neck cancers that is 5- to 25-fold increased over the general population. The ex-smoker's risk for squamous cell cancer of the head and neck begins to approach the risk in the general population twenty years after smoking cessation. The high prevalence of tobacco and alcohol use worldwide and the high association of these cancers with these substances makes them ideal targets for enhanced cancer prevention. Alcohol and tobacco are likely synergistic in causing cancer of the head and neck. Smokeless tobacco is cause of oral and pharyngeal cancers (oropharyngeal cancer). Cigar smoking is an important risk factor for oral cancers as well.

Other potential environmental carcinogens include occupational exposures such as nickel refining, exposure to textile fibers, and woodworking. Use of marijuana, especially while younger, is linked to an increase in squamous-cell carcinoma cases while other studies suggest use is not shown to be associated with oral squamous cell carcinoma, or associated with decreased squamous cell carcinoma.

Excessive consumption of processed meats and red meat were associated with increased rates of cancer of the head and neck in one study, while consumption of raw and cooked vegetables seemed to be protective. Betel nut chewing is associated with an increased risk of squamous cell cancer of the head and neck.

Recent evidence is accumulating pointing to a viral origin for some head and neck cancers. Human papillomavirus (HPV), in particular HPV16 and HPV18, is a causal factor for some head and neck squamous-cell carcinoma (HNSCC). Approximately 15 to 25% of HNSCC contain genomic DNA from HPV, and the association varies based on the site of the tumor, especially HPV-positive oropharyngeal cancer, with highest distribution in the tonsils, where HPV DNA is found in (45 to 67%) of the cases, less often in the hypopharynx (13%-25%), and least often in the oral cavity (12%-18%) and larynx (3%-7%).

Some experts estimate that while up to 50% of cancers of the tonsil may be infected with HPV, only 50% of these are likely to be caused by HPV (as opposed to the usual tobacco and alcohol causes). The role of HPV in the remaining 25-30% is not yet clear. Oral sex is not risk free and results in a significant proportion of HPV-related head and neck cancer. Positive HPV16/18 status is associated with improved prognosis over HPV-negative OSCC.

Induction of cancer can be associated for the expression of viral oncoproteins, the most important E6 and E7, or other mechanisms many of them run by the integration such as the generation of altered transcripts, disruption of tumor suppressors, high levels of DNA amplifications, interchromosomial rearrangements, or changes in DNA methylation patterns, the latter being able to find even when the virus is identified in episomes. E6 sequesters p53 to promote p53 degradation while pRb inhibits E7. p53 prevents cell growth when DNA is damaged by activating apoptosis and p21, a kinase that blocks the formation of cyclin D/Cdk4 avoiding pRb phosphorylation and thereby prevents release of E2F is a transcription factor required for activation of genes involved in cell proliferation. pRb remains bound to E2F while this action phosphorylated preventing activation of proliferation. Therefore, E6 and E7 act synergistically in triggering cell cycle progression and therefore uncontrolled proliferation by inactivating the p53 and Rb tumor suppressors.

A patient usually presents to the physician complaining of one or more of the above symptoms. The patient will typically undergo a needle biopsy of this lesion, and a histopathologic information is available, a multidisciplinary discussion of the optimal treatment strategy will be undertaken between the radiation oncologist, surgical oncologist, and medical oncologist.

Squamous-cell carcinoma is a cancer of the squamous cell—a kind of epithelial cell found in both the skin and mucous membranes. It accounts for over 90% of all head and neck cancers, including more than 90% of throat cancer. Squamous cell carcinoma is most likely to appear in males over 40 years of age with a history of heavy alcohol use coupled with smoking. The tumor marker Cyfra 21-1 may be useful in diagnosing squamous cell carcinoma of the head/neck.

Adenocarcinoma is a cancer of epithelial tissue that has glandular characteristics. Several head and neck cancers are adenocarcinomas (either of intestinal or non-intestinal cell-type).

Avoidance of recognised risk factors (as described above) is the single most effective form of prevention. Regular dental examinations may identify pre-cancerous lesions in the oral cavity. When diagnosed early, oral, head and neck cancers can be treated more easily and the chances of survival increase tremendously.

Improvements in diagnosis and local management, as well as targeted therapy, have led to improvements in quality of life and survival for people with head and neck cancer. After a histologic diagnosis has been established and tumor extent determined, the selection of appropriate treatment for a specific cancer depends on a complex array of variables, including tumor site, relative morbidity of various treatment options, patient performance and nutritional status, concomitant health problems, social and logistic factors, previous primary tumors, and patient preference. Treatment planning generally requires a multidisciplinary approach involving specialist surgeons and medical and radiation oncologists.

Several generalizations are useful in therapeutic decision making, but variations on these themes are numerous. Surgical resection and radiation therapy are the mainstays of treatment for most head and neck cancers and remain the standard of care in most cases. For small primary cancers without regional metastases (stage I or II), wide surgical excision alone or curative radiation therapy alone is used. More extensive primary tumors, or those with regional metastases (stage III or IV), planned combinations of pre- or postoperative radiation and complete surgical excision are generally used. More recently, as historical survival and control rates are recognized as less than satisfactory, there has been an emphasis on the use of various induction or concomitant chemotherapy regimens.

Many different treatments and therapies are used in the treatment of throat cancer. The type of treatment and therapies used are largely determined by the location of the cancer in the throat area and also the extent to which the cancer has spread at time of diagnosis. Patients' also have the right to decide whether or not they wish to consent to a particular treatment. For example, some may decide to not undergo radiation therapy which has serious side effects if it means they will be extending their lives by only a few months or so. Others may feel that the extra time is worth it and wish to pursue the treatments.

Surgery as a treatment is frequently used in most types of head and neck cancer. Usually the goal is to remove the cancerous cells entirely. This can be particularly tricky if the cancer is near the larynx and can result in the patient being unable to speak. Surgery is also commonly used to resect (remove) some or all of the cervical lymph nodes to prevent further spread of the disease.

CO₂ laser surgery is also another form of treatment. Transoral laser microsurgery allows surgeons to remove tumors from the voice box with no external incisions. It also allows access to tumors that are not reachable with robotic surgery. During the surgery, surgeon and pathologist work together to assess the adequacy of excision (“margin status”), minimizing the amount of normal tissue removed or damaged. This technique helps give the patient as much speech and swallowing function as possible after surgery.

Radiation therapy is the most common form of treatment. There are different forms of radiation therapy, including 3D conformal radiation therapy, intensity-modulated radiation therapy, particle beam therapy and brachytherapy, which are commonly used in the treatments of cancers of the head and neck. Most patients with head and neck cancer who are treated in the United States and Europe are treated with intensity-modulated radiation therapy using high energy photons. At higher doses, head and neck radiation is associated with thyroid dysfunction and pituitary axis dysfunction.

Chemotherapy in throat cancer is not generally used to cure the cancer as such. Instead, it is used to provide an inhospitable environment for metastases so that they will not establish in other parts of the body. Typical chemotherapy agents are a combination of paclitaxel and carboplatin. Cetuximab is also used in the treatment of throat cancer.

Docetaxel-based chemotherapy has shown a very good response in locally advanced head and neck cancer. Taxotere is the only taxane approved by US FDA for Head and neck cancer, in combination with cisplatin and fluorouracil for the induction treatment of patients with inoperable, locally advanced squamous cell carcinoma of the head and neck.

While not specifically a chemotherapy, amifostine is often administered intravenously by a chemotherapy clinic prior to a patient's IMRT radiotherapy sessions. Amifostine protects the patient's gums and salivary glands from the effects of radiation.

Photodynamic therapy may have promise in treating mucosal dysplasia and small head and neck tumors Amphinex is giving good results in early clinical trials for treatment of advanced head and neck cancer.

Targeted therapy, according to the National Cancer Institute, is “a type of treatment that uses drugs or other substances, such as monoclonal antibodies, to identify and attack specific cancer cells without harming normal cells.” Some targeted therapy used in squamous cell cancers of the head and neck include cetuximab, bevacizumab and erlotinib.

The best quality data are available for cetuximab since the 2006 publication of a randomized clinical trial comparing radiation treatment plus cetuximab versus radiation treatment alone. This study found that concurrent cetuximab and radiotherapy improves survival and locoregional disease control compared to radiotherapy alone, without a substantial increase in side effects, as would be expected with the concurrent chemoradiotherapy, which is the current gold standard treatment for advanced head and neck cancer. Whilst this study is of pivotal significance, interpretation is difficult since cetuximab-radiotherapy was not directly compared to chemoradiotherapy. The results of ongoing studies to clarify the role of cetuximab in this disease are awaited with interest.

Another study evaluated the impact of adding cetuximab to conventional chemotherapy (cisplatin) versus cisplatin alone. This study found no improvement in survival or disease-free survival with the addition of cetuximab to the conventional chemotherapy. However, another study which completed in March 2007 found that there was an improvement in survival. A 2010 review concluded that the combination of cetuximab and platin/5-fluorouracil should be considered the current standard first-line regimen.

Gendicine is a gene therapy that employs an adenovirus to deliver the tumor suppressor gene p53 to cells. It was approved in China in 2003 for the treatment of head and neck squamous cell carcinoma. Head and neck cancer clinical trials employing bevacizumab, an inhibitor of the angiogenesis receptor VEGF, were recruiting patients as of March 2007.

Erlotinib is an oral EGFR inhibitor, and was found in one Phase II clinical trial to retard disease progression. Scientific evidence for the effectiveness of erlotinib is otherwise lacking to this point. Clinical trials have shown no benefit in unselected patients with advanced disease (23).

Although early-stage head and neck cancers (especially laryngeal and oral cavity) have high cure rates, up to 50% of head and neck cancer patients present with advanced disease. Cure rates decrease in locally advanced cases, whose probability of cure is inversely related to tumor size and even more so to the extent of regional node involvement.

Consensus panels in America (AJCC) and Europe (UICC) have established staging systems for head and neck squamous-cell cancers. These staging systems attempt to standardize clinical trial criteria for research studies, and attempt to define prognostic categories of disease. Squamous cell cancers of the head and neck are staged according to the TNM classification system, where T is the size and configuration of the tumor, N is the presence or absence of lymph node metastases, and M is the presence or absence of distant metastases. The T, N, and M characteristics are combined to produce a “stage” of the cancer, from I to IVB.

Survival advantages provided by new treatment modalities have been undermined by the significant percentage of patients cured of head and neck squamous cell carcinoma (HNSCC) who subsequently develop second primary tumors. The incidence of second primary tumors ranges in studies from 9% to 23% at 20 years. Approximately 30% of OSCC patients will have recurrent disease. Second primary tumors are the major threat to long-term survival after successful therapy of early-stage HNSCC. Their high incidence results from the same carcinogenic exposure responsible for the initial primary process, called field cancerization.

### II. THYMOL AND THYMOL DERIVATIVES

Thymol (also known as 2-isopropyl-5-methylphenol, IPMP) is a natural monoterpene phenol derivative of cymene, C₁₀H₁₄O, isomeric with carvacrol, found in oil of thyme, and extracted from Thymus vulgaris (common thyme) and various other kinds of plants as a white crystalline substance of a pleasant aromatic odor and strong antiseptic properties. Thymol also provides the distinctive, strong flavor of the culinary herb thyme, also produced from T. vulgaris.

Thymol is part of a naturally occurring class of compounds known as biocides, with strong antimicrobial attributes when used alone or with other biocides such as carvacrol. In addition, naturally occurring biocidal agents such as thymol can reduce bacterial resistance to common drugs such as penicillin. Numerous studies have demonstrated the antimicrobial effects of thymol, ranging from inducing antibiotic susceptibility in drug-resistant pathogens to powerful antioxidant properties. Research demonstrates that naturally occurring biocides such as thymol and carvacrol reduce bacterial resistance to antibiotics through a synergistic effect, and thymol has been shown to be an effective fungicide, particularly against fluconazole-resistant strains. This is especially relevant to Candida infections. Compounds in the essential oils of one type of oregano have demonstrated antimutagenic effects, and in particular carvacrol (isomeric with thymol) and thymol were demonstrated to have a strong antimutagenic effect. In addition, there is evidence that thymol has antitumor properties. Though the exact mechanism is unknown, some evidence suggests thymol effects at least some of its biocidal properties by membrane disruption. Thymol has been shown to act as a positive allosteric modulator of GABA_{A} in vitro. Thymol is chemically related to the anesthetic propofol.

Thymol is only slightly soluble in water at neutral pH, but it is extremely soluble in alcohols and other organic solvents. It is also soluble in strongly alkaline aqueous solutions due to deprotonation of the phenol. Synonyms include isopropyl-m-cresol, cymene, 1-methyl-3-4-isopropylbenzene, 2-1-isopropyl-4-methylbenzene; 3-p-cymene, 3-methyl-6-isopropylphenol, 5-methyl-2-(1-methylethyl)phenol, 5-methyl-2-isopropyl-1-phenol, 5-methyl-2-isopropylphenol, 6-isopropyl-3-methylphenol, 6-isopropyl-m-cresol, Apiguard, NSC 11215, NSC 47821, NSC 49142, thyme camphor, m-thymol, and p-cymen-3-ol. Thymol has a refractive index of 1.5208 and an experimental dissociation exponent (pKa) of 10.59±0.10. Thymol absorbs maximum UV radiation at 274 nm.

The Ancient Egyptians used thymol and carvacrol in the form of a preparation from the thyme plant (a member of the mint family) to preserve mummies Thymol and carvacrol are now known to kill bacteria and fungi, making thyme well suited for such purposes. In Ancient Greece, thyme was widely used for its aromatic qualities, being burned as incense in sacred temples. Thyme was also a symbol of courage and admiration, with the phrase “the smell of thyme” being a saying that reflected praise unto its subject. Thyme's association with bravery continued throughout medieval times, when it was a ritual for women to give their knights a scarf that had a sprig of thyme placed over an embroidered bee.

Since the 16th century, thyme oil has been used for its antiseptic properties, both as mouthwash and for topical application. The bee balms Monarda fistulosa and Monarda didyma, North American wildflowers, are natural sources of thymol. The Blackfoot Native Americans recognized these plants' strong antiseptic action, and used poultices of the plants for skin infections and minor wounds. A tisane made from them was also used to treat mouth and throat infections caused by dental caries and gingivitis.

Thymol has antimicrobial activity because of its phenolic structure, and has shown antibacterial activity against bacterial strains including Aeromonas hydrophila and Staphylococcus aureus. In addition, thymol demonstrates considerable post antibacterial effect against some microorganisms. This antibacterial activity is caused by inhibiting growth and lactate production, and by decreasing cellular glucose uptake.

Thymol has been used in alcohol solutions and in dusting powders for the treatment of tinea or ringworm infections, and was used in the United States to treat hookworm infections. People of the Middle East continue to use za′atar, a delicacy made with large amounts of Thyme, to reduce and eliminate internal parasites.

It is also used as a preservative in halothane, an anaesthetic, and as an antiseptic in mouthwash. When used to reduce plaque and gingivitis, thymol has been found to be more effective when used in combination with chlorhexidine than when used purely by itself. Thymol is also the active antiseptic ingredient in some toothpastes, such as Euthymol.

The antifungal nature of thymol is caused by thymol's ability to alter the hyphal morphology and cause hyphal aggregates, resulting in reduced hyphal diameters and lyses of hyphal wall. Additionally, thymol is lipophilic, enabling it to interact with the cell membrane of fungus cells, altering cell membrane permeability permitting the loss of macromolecules. Recent medical research on rats concludes that “Thyme extract had relaxing effects on organs possessing β₂-receptors (uterus and trachea).” In a 1994 report released by five major cigarette companies, thymol was listed as one of 599 additives to cigarettes.

Thymol has been used to successfully control varroa mites and prevent fermentation and the growth of mold in bee colonies, methods developed by beekeeper R.O.B. Manley. Thymol is also used as a rapidly degrading, non-persisting pesticide.

Derivatives of thymol and carvacrol with increased antimicrobial activities have been developed. The preparation of methacrylic and p-styrenesulfonic acid esters of thymol could lead to less toxic macromolecular biocides, which can be attached to a polymeric backbone.

Thymol can also be used as a medical disinfectant and general purpose disinfectant. For instance, thymol is used as the starting material used to synthesize biclotymol. Thymol can be brominated to Bromthymol, which is a recognized anthelmintic drug.

### III. ORAL ADMINISTRATION FORMS

The present disclosure contemplates delivery of an oral dosage form to a subject that includes thymol. In one embodiment, the thymol is in the form of an injectable solution. Alternatively, the thymol is delivered in an oral-topical form, such as a mouthwash and an effervescent tablet or a powder or granules dissolving easily in an aqueous solution, such as a mouthwash, which tablet, powder or granules comprise thymol or a derivative thereof, whereby the combination is intended for decreasing the risk of or treat oral cancer. The effervescent tablet, powder or granules can be dissolved either in the mouthwash or in a glass of water prior to use.

The mouthwash may contain a liquid medium including ethanol and a fluorine compound, such as fluoride. It may also lack alcohol.

In order to obtain a desired appearance and flavor, aspects of the delivery form may further include edulcorant, solubilizer, a bacteriocide and colorant ingredients.

In order to facilitate the delivery form's use and stability, aspects may further include pH adjusting ingredients, preservatives and antifoaming ingredients.

These ingredients may be included in an aqueous-based media, so that active agents may be conducted to the oral cavity.

The group of ingredients having a flavorizing effect includes, but is not limited to, Zinc citrate, Zinc chloride, Tutti frutti, Menthol, Methyl salicylate, eucalyptus oil, spearmint oil, and peppermint oil. In one preferred aspect, a variety of ingredients with a flavorizing effect may be used to allow for a broad range of flavor options.

The ingredient group having a flavorizing effect can be found in the formulation in about 0.01% to about 1% of the volume, or preferably in about 0.05% to about 0.5% of the volume, or preferably in about 0.075% to about 0.3% of the volume, or preferably in about 0.15% of the volume of the whole formulation.

The group of ingredients having a bacteriocide effect includes, but is not limited to, Cetylpyridinium chloride, Delmopinol, Benzalkonium chloride, Sodium bicarbonate, Chlorhexidine gluconate, Chlorhexidine digluconate, Chlorine dioxide (Sodium chlorite/Sodium chlorate), Triclosan, Polyhexamethylene biguanide chlorhydrate, Sanguinaria Canadensis, Propolis, Aloe vera, Sage (Salvia officinalis), Lemon (Citrus limon), Pine (Pinus sylvestris), Echinacea (Echinacea purpurea and angustifolia), Rathany (Krameria trianda) and Cheeseweed mallow (Malva parviflora L.). In one preferred aspect, the bactericide ingredient comprises Cetylpyridinium chloride.

The ingredient group having a bacteriocide effect can be found in the formulation in about 0.01% to about 0.5% of the volume, or preferably from about 0.02% to about 0.25% of the volume, or preferably in about 0.03% to about 0.1% of the volume, or preferably in about 0.05% of the volume of the whole formulation.

The group of ingredients used to obtain ideal appearance and flavor includes, but is not limited to, ingredients with an edulcorant, solubilizer, or a colorant effect. The

The ingredient group having an edulcorant effect includes, but is not limited to, Sodium saccharine, Sorbitol, Xylitol, Aspartame, Sodium cyclamate, and Stevia. In one preferred aspect, the ingredients having an edulcorant effect comprise Sodium saccharine or Sorbitol.

The group of ingredients having an edulcorant effect, more specifically Sodium saccharine, may be present in the formulation in a quantity of about 0.01% to about 1% of the volume, or preferably in about 0.02% to about 0.5% of the volume, or preferably in about 0.04% to about 0.1% of the volume, or preferably in about 0.05% of the volume of the whole formulation.

The group of ingredients having an edulcorant effect, more specifically Sorbitol, may be present in the formulation in a quantity of about 0.5% to about 20% of the volume, or preferably in about 1% to about 15% of the volume, or preferably in about 3% to about 10% of the volume, or preferably in about 4% of the volume of the whole formulation.

The ingredient group having a solubilizer effect includes, but is not limited to, Polisorbate 20 (Tween 20), Propylene glycol, Polyoxyl 40 and a solubilizer that mixes Propylene glycol, Polyethylene glycol and water. In one preferred aspect, the solubilizer ingredient comprises Polisorbate 20.

The group of ingredients having a solubilizer effect may be present in the formulation in a quantity of about 0.05% to about 2% of the volume, or preferably in about 0.1% to about 1.5% of the volume, or preferably in about 0.25% to about 1% of the volume, or preferably in about 0.5% of the volume of the whole formulation.

The group of ingredients having a colorant effect includes, but is not limited to, all colorants, for food or oral hygiene products. In one preferred aspect, different ingredients with a colorant effect may be used to provide the product with a range of color options.

The group of ingredients having a colorant effect may be pre-sent in the formulation in a quantity of about 0.0001% to about 1% of the volume, or preferably in about 0.0005% to about 0.1% of the volume, or preferably in about 0.001% to about 0.01% of the volume, or preferably in about 0.002% of the volume of the whole formulation.

The group of ingredients facilitating the use and the stability of the product includes, but is not limited to, pH adjusters, preservatives and antifoamer ingredients.

The group of pH adjuster includes, but is not limited to, boric acid and citric acid. In one preferred aspect, the pH adjuster comprises citric acid.

The group of ingredients having a pH adjusting effect may be present in the formulation in a quantity of about 0.01% to about 1% of the volume, or preferably in about 0.05% to about 0.5% of the volume, or preferably in about 0.1% to about 0.25% of the volume, or preferably in about 0.15% of the volume of the whole formulation.

The group of ingredient having a preservative effect includes, but is not limited to, Sodium benzoate, Nipagin or Methyl paraben, Benzoic acid, Formaldehyde, Thymol and Nipazol or Isopropyl paraben. In one preferred aspect, the preservatives comprise Sodium benzoate or Methyl paraben.

The group of ingredients having a preservative effect, more specifically Methyl paraben, may be present in the formulation in a quantity of about 0.01% to about 0.5% of the volume, or preferably in about 0.02% to about 0.25% of the volume, or preferably in about 0.03% to about 0.1% of the volume, or preferably in about 0.05% of the volume of the whole formulation.

The group of ingredients having a preservative effect, more specifically Sodium benzoate, may be present in the formulation in a quantity of about 0.01% to about 1.0% of the volume, or preferably in about 0.05% to about 0.5% of the volume, or preferably in about 0.1% to about 0.25% of the volume, or preferably in about 0.15% of the volume of the whole formulation.

The group of antifoamer ingredients includes, but is not limited to, Dimethicone. The group of antifoamer ingredients may be present in a quantity of about 0.005% to about 0.5% of the volume, or preferably in about 0.075% to about 0.25% of the volume, or preferably in about 0.01% to about 0.1% of the volume, or preferably in about 0.03% of the volume of the whole formulation.

The group of ingredients enabling active agents to be conducted to the oral cavity in an aqueous-based solubilizer media includes, but is not limited to, demineralized water, distilled water, deionized water, and mineral water. In one preferred aspect, the aqueous-based solubilizer media comprises demineralized water; The delivery form may also include ethanol, having an ethanol content varying within 0.7% by volume and 20% by volume, or 1.0 weight % to 30 weight %, 15 weight % to 25 weight %, or 1.0 weight % to 5 weight % of the entire mouthwash.

The product may, for example, be added into vessels, such as bottles, containing a unit dose of both the mouthwash or the effervescent tablet (or powder or granules), where the mouthwash suitably is stored in the bottle and the effervescent tablet (or powder or granules) is stored in a single use package. These are easily carried by the subject, for example, in a pocket or a handbag. Such vessels may be further comprises within a kit that includes various other elements including carrier liquids, mixing containers and/or instructions for use of the compositions.

According to a particularly preferred alternative, the above described liquid composition is a mouthwash, which in turn particularly is selected from ethanol-containing mouthwashes.

According to an embodiment of the disclosure, the mouthwash is based on water, excluding alcohol, and comprising one or more aromatic agents, particularly for improving the taste of the mouthwash or the breath of the subject, and optionally one or more antimicrobial agents. Further additives may also be included, such as agents reducing the sensitivity of the nerve endings of teeth (e.g., potassium nitrate), and/or xylitol.

The amount of mouthwash administered is preferably 10 to 30 ml per unit dose, more preferably about 20 ml per unit dose, and may be dosed 1 to 2 times a day (e.g., once in the morning and once in the evening). The unit dose of the mouthwash is generally used by mixing in the mouth for about 30 seconds. However, the desired effect is obtained at least to some extent already by a mixing time of 10 seconds, and no harmful effect takes place with a mixing time of 1 minute.

### IV. TREATING CANCERS

Provided herein are methods for treating or delaying progression of oral cancer in an individual comprising administering to the individual an effective amount of thymol or a thymol derivative to a subject who is at risk of or had been diagnosed as having oral cancer.

In some embodiments, the treatment results in prevention of disease or a sustained response in the individual, including after cessation of the treatment. The methods described herein may find use in treating recurrent cancers, including cancers in patients that have previously received other therapies—with or without response. In some embodiments, the individual is a human.

Examples of cancers contemplated for treatment include squamous cell carcinomas and adenocarcinomas, but the inventor also has data showing that thymol has anti-tumor effects against all types of solid tumors (breast, lung, prostate, colon, cervical). However head and neck cancer is 90% of squamous cell origin.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

The therapy provided herein comprises administration of thymol or a derivative thereof to an oral cavity. The appropriate dosage and timing of administration depends on the type of disease to be treated, severity and course of the disease, the clinical condition of the individual, the individual's clinical history and response to the treatment, and the discretion of the attending physician. For example, the therapeutically effective amount of may be in the range of about 1 to 100 mM, about 1-50 mM, about 1-20 mM, about 1-10 mM, or about 2-5 mM. The dose may be administered as a single dose or as multiple doses (e.g., 2 or 3 doses). The progress of this therapy is easily monitored by conventional techniques.

In certain embodiments, the tumor being treated may not, at least initially, be resectable. Treatments may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single administration but may comprise continuous infusion over a set period of time.

In some embodiments, thymol or its derivatives may be administered in combination with at least one additional therapeutic. The additional therapy may be a cancer therapy such as radiation therapy, surgery, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional cancer therapy is the administration of a small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional cancer therapy is radiation therapy. In some embodiments, the additional cancer therapy is surgery. In some embodiments, the additional cancer therapy is a combination of radiation therapy and surgery. In some embodiments, the additional cancer therapy is gamma irradiation. In some embodiments, the additional cancer therapy is therapy targeting PBK/AKT/mTOR pathway, HSP90 inhibitor, tubulin inhibitor, apoptosis inhibitor, and/or chemopreventative agent. The additional cancer therapy may be one or more of the chemotherapeutic agents known in the art.

Various combinations may also be employed. For the example below the thymol or derivative is “A” and an additional cancer therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | |
| A/B/A/B | A/B/B/A | B/B/A/A | B/A/B/A | B/A/A/B | |
| A/A/A/B | B/A/A/A | A/B/A/A | A/A/B/A | | |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

Chemotherapy.

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

Radiotherapy.

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Immunotherapy.

The skilled artisan will understand that immunotherapies may be used in combination or in conjunction with the methods described herein. In the context of cancer treatment, immunotherapeutics generally rely on the use of immune effector cells and molecules to target and destroy cancer cells. Rituximab (RITUXAN®) is an example of an immunotherapy. The immune effector may be, for example, an antibody specific for a marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment. As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

### Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Anti-Cancer Agents.

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. EXAMPLES

The following examples are included to further illustrate various aspects of the disclosure. It should be appreciated by those of skill in the art that the techniques disclosed in the examples which follow represent techniques and/or compositions discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

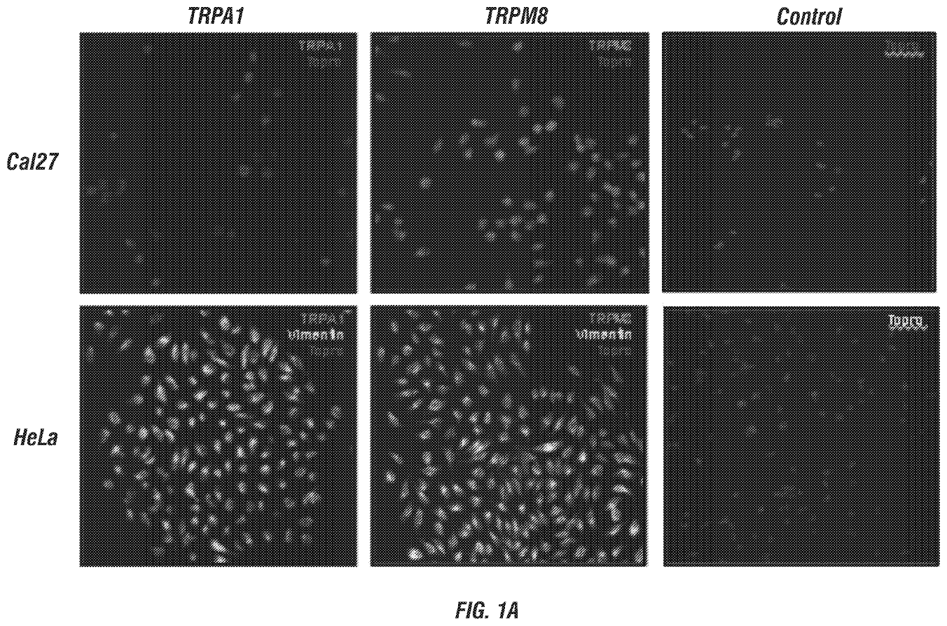

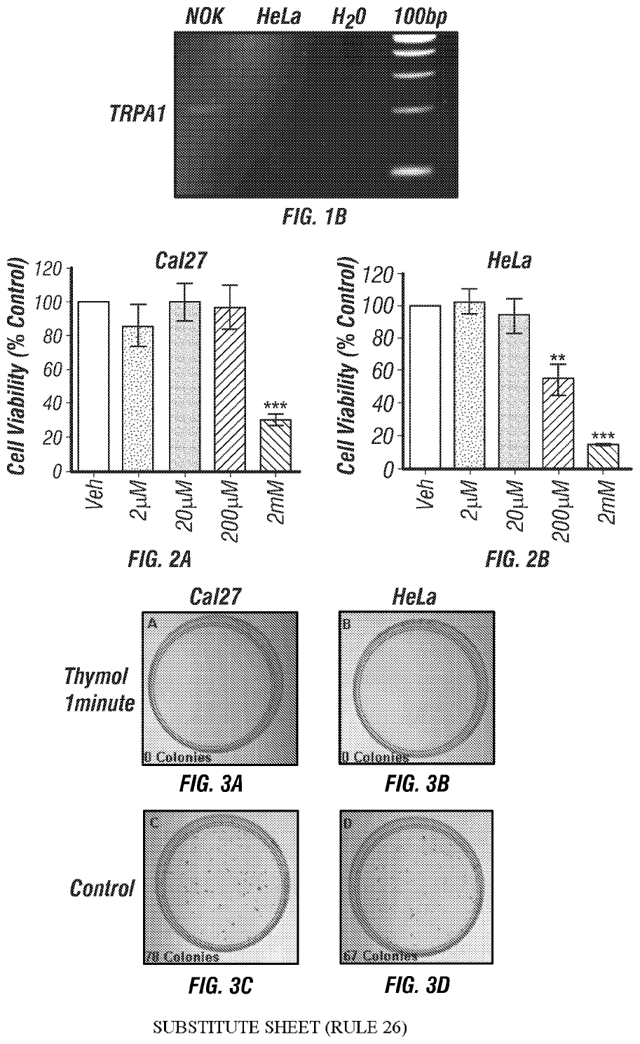

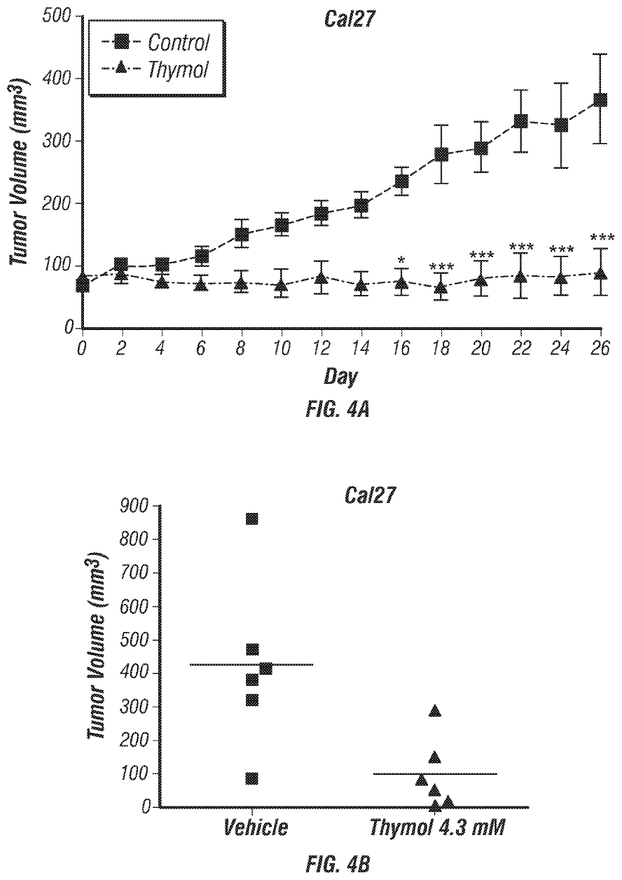

Preliminary immunohistochemical studies of TRP channel expression in cancer cell lines determined that TRPA1 is expressed in the OSCC cell line, Ca127 (FIG. 1A; top row), but not in the cervical cancer cell line, HeLa (FIG. 1A; bottom row). RT-PCR confirmed no TRPA1 mRNA expression in HeLa cells. TRPM8 expression was found via immunohistochemistry in both Ca127 and HeLa cells (FIG. 1A; middle panels). Cell viability assays demonstrated that thymol has significant anti-proliferative effects in vitro in both Ca127 and HeLa cells (IC₅₀ 400 μM) regardless of TRP channel expression (FIGS. 2A-B; p<0.001). Clonogenic assays determined that a one minute exposure of cancer cells abolished colony formation for a period of 10 days (FIGS. 3A-D).

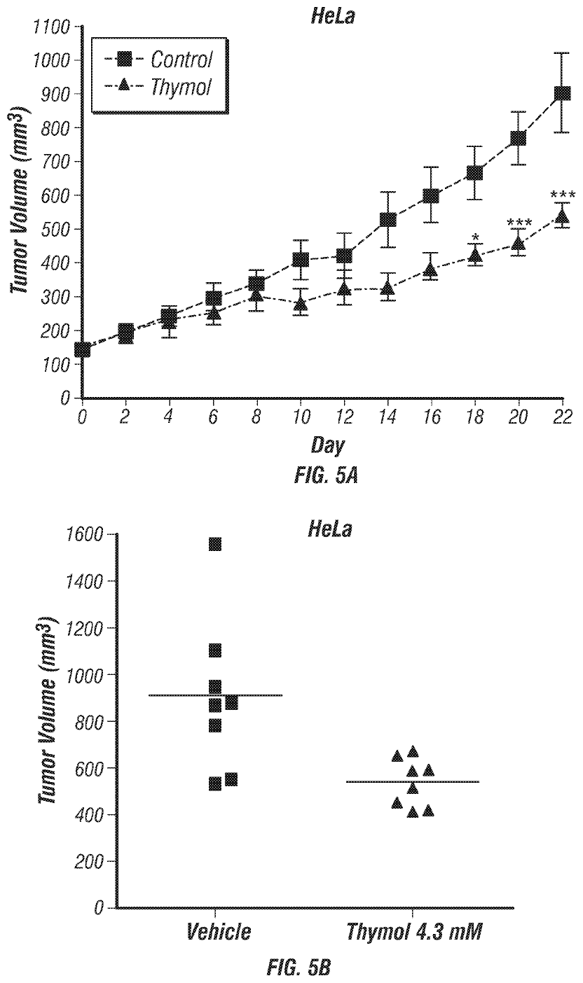

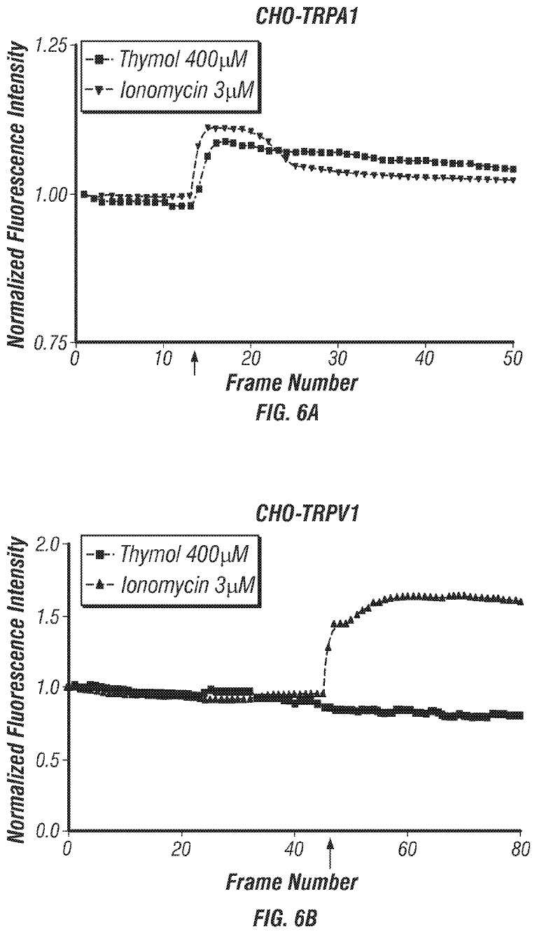

The inventor then evaluated anti-tumor effects of thymol in mouse xenograft models. Ca127-derived tumors were allowed to grow to approximately 100 mm³ and then treated every other day with thymol (4.3 mM) via intra-tumor injection for 26 days. By day 16, a significant reduction in tumor volume was detected compared to control (FIG. 4A; p<0.001) with an average tumor volume of 90 mm³ compared to control, 400 mm³ (FIG. 4B) Similarly, HeLa-derived tumors were allowed to grow to approximately 100 mm³ and then treated with thymol (4.3 mM) via intra-tumor injection every other day for 22 days. By day 18 a significant reduction in tumor volume was detected that remained through day 22 (FIG. 5A; p<0.001). The average tumor volume of thymol treated tumors was 500 mm³ compared to control, 900 mm³ (FIG. 5B). Inhibition of tumor growth was more efficacious in the OSCC, Ca127-derived, tumors.

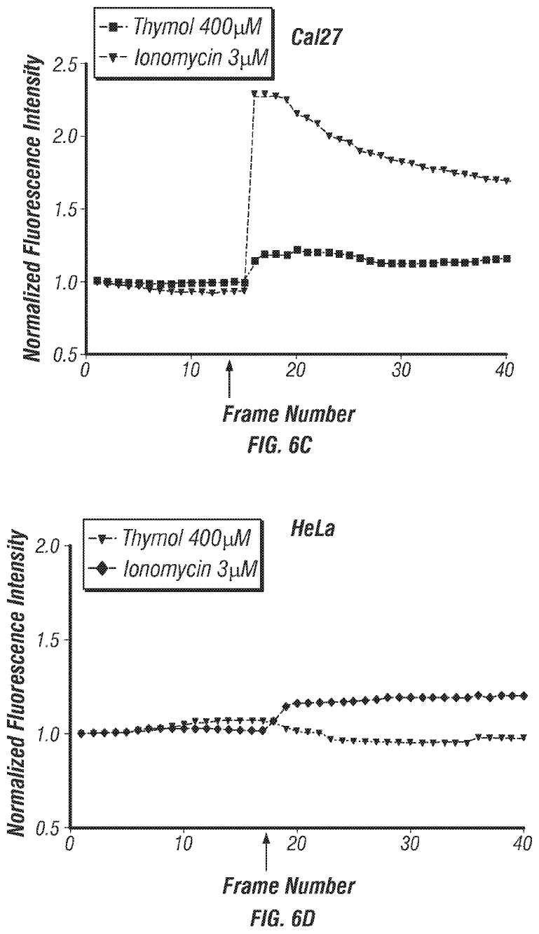

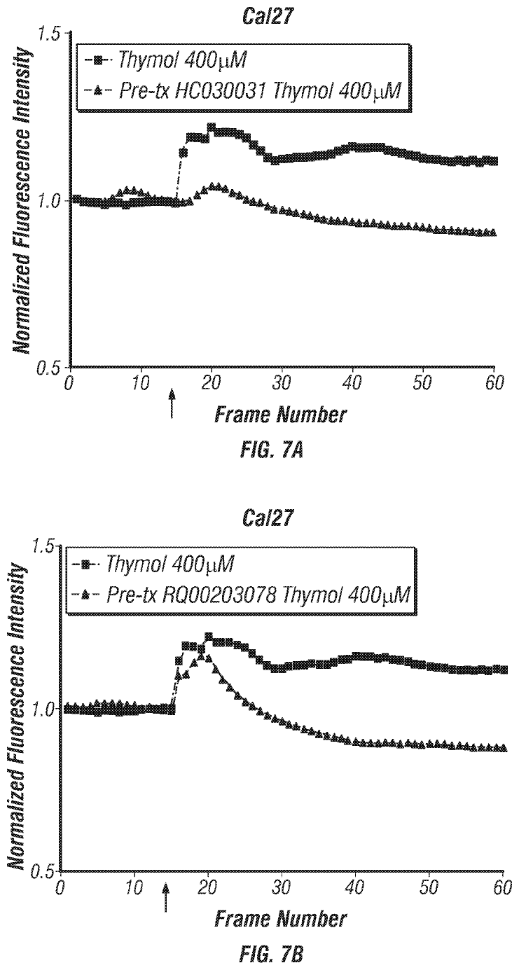

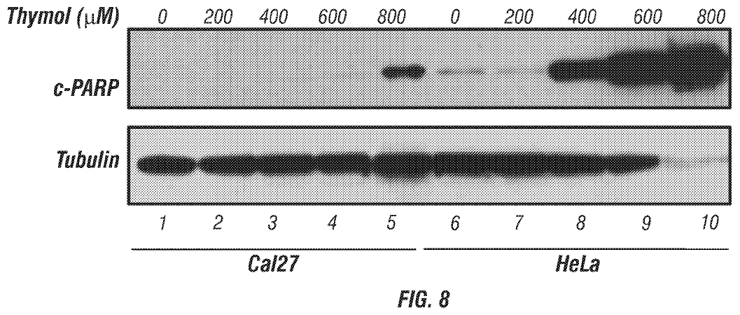

Calcium imaging confirmed that thymol activates TRPA1 channels (FIG. 6A) but not TRPV1 channels (FIG. 6B). Likewise Ca127 cells, expressing TRPA1, had a small calcium influx when treated with thymol (400 μM) whereas HeLa cells, negative for TRPA1, did not respond to thymol treatments. This indicates that while TRPA1 channels are active in Ca127 cells, the anti-tumor effects of thymol are most likely not due to TRPA1 activity. Pre-treatment of Ca127 cells with the TRPA1 inhibitor HC030031 nearly abolished all calcium influx in response to thymol (FIG. 7A). Pre-treatment with the TRPM8 inhibitor RQ00203078 reduced calcium influx but the effect was not as dramatic as that seen with the TRPA1 inhibitor (FIG. 7B) indicating that TRPA1 is the primary TRP channel that is activated by thymol. MTS assays in which Ca127 cells were pre-treated with TRPA1 and TRPM8 inhibitors revealed that TRP channel inhibition had no effect on the anti-proliferative effects of thymol indicating that thymol anti-cancer effects are most likely due to a different mechanism than TRP channel activation (data not shown). Therefore, the inventor evaluated if thymol induces apoptosis in cancer cell lines, indeed, a dose-dependent activation of cleaved PARP was demonstrated using western blot analysis in both Ca127 and HeLa cells (FIG. 8). Therefore, the inventor concludes that thymol has significant anti-tumor effects against oral cancers via an unidentified mechanism that does not involve TRP channel activity.

The inventor is now generating thymol analogs using structure-activity-relationship studies to move away from the TRP channel activity and develop more potent anti-cancer activity. Given that oral cancer has a 30% recurrence rate, HPV associated oral cancers are on the rise, and the fact that there has never been a chemopreventative agent for oral cancer, developing thymol analogs to both treat and prevent oral cancers is of the utmost importance. The disclosure therefore contemplates developing thymol analogs to be delivered via a mouth rinse for prevention or delivered via intra-tumor injection and/or systemic administration for the treatment of existing oral cancers. This technology offers the first easy-to-use, over-the-counter, means of preventing oral cancer. Furthermore, no effective therapies aimed at treating advanced OSCC have been developed in nearly 40 years. Therefore, prevention is of the utmost importance. This technology would prevent tumor development and also prevent progression to advanced disease which has a mere 40% 5-year survival rate.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit and scope of the disclosure as defined by the appended claims.

### VI. REFERENCES

The following references, to the extent that they provide exemplary procedural or other details supplementary to those set forth herein, are specifically incorporated herein by reference.
1. 2015. Cancer Facts and Figures. American Cancer Society.
2. Newman, D. J., and Cragg, G. M. Natural products as sources of new drugs over the 30 years from 1981 to 2010. J Nat Prod 75:311-335.
3. Pan, L., Chai, H. B., and Kinghorn, A. D. Discovery of new anticancer agents from higher plants. Front Biosci (Schol Ed) 4:142-156.
4. Robles, A. J., Peng, J., Hartley, R. M., Lee, B., and Mooberry, S. L. Melampodium leucanthum, a source of cytotoxic sesquiterpenes with antimitotic activities. J Nat Prod 78:388-395.
5. El Babili, F., Bouajila, J., Souchard, J. P., Bertrand, C., Bellvert, F., Fouraste, I., Moulis, C., and Valentin, A. Oregano: chemical analysis and evaluation of its antimalarial, antioxidant, and cytotoxic activities. J Food Sci 76:C512-518.
6. Pandey, S. K., Upadhyay, S., and Tripathi, A. K. 2009. Insecticidal and repellent activities of thymol from the essential oil of Trachyspermum ammi (Linn) Sprague seeds against Anopheles stephensi. Parasitol Res 105:507-512.
7. Miyazawa, N., Fujita, A., and Kubota, K. Aroma character impact compounds in Kinokuni mandarin orange (Citrus kinokuni) compared with Satsuma mandarin orange (Citrus unshiu). Biosci Biotechnol Biochem 74:835-842.
8. Conforti, F., Statti, G., Uzunov, D., and Menichini, F. 2006. Comparative chemical composition and antioxidant activities of wild and cultivated Laurus nobilis L. leaves and Foeniculum vulgare subsp. piperitum (Ucria) coutinho seeds. Biol Pharm Bull 29:2056-2064.
9. Ozcan, M. M., and Chalchat, J. C. 2008. Chemical composition and antifungal activity of rosemary (Rosmarinus officinalis L.) oil from Turkey. Int J Food Sci Nutr 59:691-698.
10. Chang, H. T., Hsu, S. S., Chou, C. T., Cheng, J. S., Wang, J. L., Lin, K. L., Fang, Y. C., Chen, W. C., Chien, J. M., Lu, T., et al. Effect of thymol on Ca2+ homeostasis and viability in MG63 human osteosarcoma cells. Pharmacology 88:201-212.
11. Mohammed, M. J., and Al-Bayati, F. A. 2009. Isolation and identification of antibacterial compounds from Thymus kotschyanus aerial parts and Dianthus caryophyllus flower buds. Phytomedicine 16:632-637.
12. Cosentino, S., Tuberoso, C. I., Pisano, B., Satta, M., Mascia, V., Arzedi, E., and Palmas, F. 1999. In-vitro antimicrobial activity and chemical composition of Sardinian Thymus essential oils. Lett Appl Microbiol 29:130-135.
13. Ahmad, A., Khan, A., Akhtar, F., Yousuf, S., Xess, I., Khan, L. A., and Manzoor, N. Fungicidal activity of thymol and carvacrol by disrupting ergosterol biosynthesis and membrane integrity against Candida. Eur J Clin Microbiol Infect Dis 30:41-50.
14. Lee, S. P., Buber, M. T., Yang, Q., Cerne, R., Cortes, R. Y., Sprous, D. G., and Bryant, R. W. 2008. Thymol and related alkyl phenols activate the hTRPA1 channel. Br J Pharmacol 153:1739-1749.
15. Baraldi, P. G., Preti, D., Materazzi, S., and Geppetti, P. Transient receptor potential ankyrin 1 (TRPA1) channel as emerging target for novel analgesics and anti-inflammatory agents. J Med Chem 53:5085-5107.
16. Ortar, G., Morera, L., Moriello, A. S., Morera, E., Nalli, M., Di Marzo, V., and De Petrocellis, L. Modulation of thermo-transient receptor potential (thermo-TRP) channels by thymol-based compounds. Bioorg Med Chem Lett 22:3535-3539.
17. Gonzales, C. B., Kirma, N. B., De La Chapa, J. J., Chen, R., Henry, M. A., Luo, S., and Hargreaves, K. M. 2014. Vanilloids induce oral cancer apoptosis independent of TRPV1. Oral Oncol 50:437-447.
18. Mergler, S., Strowski, M. Z., Kaiser, S., Plath, T., Giesecke, Y., Neumann, M., Hosokawa, H., Kobayashi, S., Langrehr, J., Neuhaus, P., et al. 2007. Transient receptor potential channel TRPM8 agonists stimulate calcium influx and neurotensin secretion in neuroendocrine tumor cells. Neuroendocrinology 85:81-92.
19. Prevarskaya, N., Zhang, L., and Barritt, G. 2007. TRP channels in cancer. Biochim Biophys Acta 1772:937-946.
20. Sanchez, M. G., Sanchez, A. M., Collado, B., Malagarie-Cazenave, S., Olea, N., Carmena, M. J., Prieto, J. C., and Diaz-Laviada, I. I. 2005. Expression of the transient receptor potential vanilloid 1 (TRPV1) in LNCaP and PC-3 prostate cancer cells and in human prostate tissue. Eur J Pharmacol 515:20-27.
21. Wissenbach, U., Niemeyer, B., Himmerkus, N., Fixemer, T., Bonkhoff, H., and Flockerzi, V. 2004. TRPV6 and prostate cancer: cancer growth beyond the prostate correlates with increased TRPV6 Ca2+ channel expression. Biochem Biophys Res Commun 322:1359-1363.
22. Hsu, S. S., Lin, K. L., Chou, C. T., Chiang, A. J., Liang, W. Z., Chang, H. T., Tsai, J. Y., Liao, W. C., Huang, F. D., Huang, J. K., et al. Effect of thymol on Ca2+ homeostasis and viability in human glioblastoma cells. Eur J Pharmacol 670:85-91.
23. Massarelli et al., Phase II trial of everolimus and erlotinib in patients with platinum-resistant recurrent and/or metastatic head and neck squamous cell carcinoma. 2015 Ann Oncol. 26(7): 1476-80.

1. A method of preventing or treating an oral cancer in a subject comprising administering isolated thymol or derivative thereof to an oral cavity of said subject.

2. The method of claim 1, wherein said oral cancer is a squamous cell carcinoma or adenocarcinoma.

3. The method of claim 1, wherein said subject is a non-human mammal.

4. The method of claim 1, wherein said subject is a human.

5. The method of claim 4, wherein said human subject partakes in one or more of smoking, chewing tobacco or consuming alcoholic beverages, such as on regular/chronic basis.

6. The method of claim 1, wherein said thymol or derivative thereof is administered in a mouthwash, such as a mouthwash comprising one or more of a flavorant, a preservative, fluoride, or an anti-bacterial/anti-fungal agent, and/or may lack alcohol.

7. (canceled)

8. The method of claim 1, wherein thymol or derivative thereof is administered at about 1 to 100 mM, about 1-50 mM, about 1-20 mM, about 1-10 mM, or about 2-5 mM.

9. The method of claim 1, wherein thymol or derivative thereof is administered more than once, such as daily, twice daily, or every other day.

10. (canceled)

11. The method of claim 1, wherein the subject is known to have an oral HPV infection.

12. The method of claim 1, wherein thymol or derivative thereof is injected into a tumor.

13. The method of claim 1, further comprising administering to said subject a second cancer therapy, such as chemotherapy, radiotherapy, immunotherapy, toxin therapy, phototherapy, cryotherapy or surgery.

14. (canceled)

15. The method of claim 1, wherein one or more of cancer development, cancer recurrence, cancer metastasis, cancer growth or cancer progression is inhibited.

16-19. (canceled)

20. The method of claim 1, wherein one or more of tumor size, tumor invasion, tumor spread, tumor recurrence or patient discomfort is reduced.

21. The method of claim 1, wherein an unresectable tumor is rendered resectable.

22. The method of claim 1, wherein said subject's survival is increased.

23. The method of claim 1, wherein said subject has previously been diagnosed with oral cancer.

24. The method of claim 1, wherein said subject has not previously been diagnosed with oral cancer.

25. The method of claim 1, wherein thymol or derivative thereof is delivered in a tablet, such as an effervescent tablet.

26. A composition selected from:
(a) mouthwash comprising thymol and a flavorant, wherein said flavorant is not thymol;
(b) an effervescent tablet comprising thymol, and optionally including a flavorant, wherein said flavorant is not thymol; and
(c) an aqueously soluble powder comprising thymol, and optionally including a flavorant, wherein said flavorant is not thymol.

27-28. (canceled)

29. A kit comprising a mouthwash, effervescent tablet or powder comprising thymol and a flavorant, wherein said flavorant is not thymol.

30. (canceled)
